SEQUENCE LISTING (20 sequences)

SEQ ID NO: 1 - protein, 99 aa, synthetic construct
MAHHHQGPQRGIRATAGVLCFLGTSTSDPSTDTSRESRSTRQTPRPLSMSKPTTGSPTADNTRTSSATSPTRSLGAPKGCYVFRGQTRTSSLPGEKEGS

SEQ ID NO: 2 - protein, 119 aa, synthetic construct
MAHHHQSPQSGIRTTAGVLCFLGTSTGPFSGLDKGEPVIEADAAALDHDKAYDRQLDGGDNLYLKYNHADAEFQERLKEDTSCGGNLGRAVFQANKRVPEPLGLVEKPVKTAPGKKRPG

SEQ ID NO: 3 - protein, 77 aa, synthetic construct
MAHHHPSPQSGIRTTAGVSCFLGTSTPDPSTDSTRECRSTRQTPRPSSTTKPTTTARQRRQPVPQVQPRRRGVSGAP

SEQ ID NO: 4 - protein, 77 aa, synthetic construct
MAHHHQSPQGGIRTTAGVLCFLGTSTSDPSTDSTRESPSTRQTPRPSSSTKPTAGNLTVETTRISSTTRRRGVSGAP

SEQ ID NO: 5 - protein, 99 aa, synthetic construct
MAHHHQSPQSGIRTTAGVLCFLGTSTSDPSTDSTRESRSTRQTPRPSSTTKPTTDSSTAETTRTSSTTTPTRSFRSALKKIRHLGATSDEQSSGEKEGS

SEQ ID NO: 6 - protein, 99 aa, synthetic construct
MEPRNPKPTSKSRTTAGVWCFLATSTSDPSTDSTRGSPSTRRMQRPSSTTRPTTSSSKRVTIRTCGITTPTPSFRSVCKKIRLLGATRTSSLPGEKEGS

SEQ ID NO: 7 - protein, 87 aa, synthetic construct
MEPRNPKPTSKSRTTAGVWCFLATSTSDPSTDSTRGSPSTRRMQRPSSTTRPTTSSSKRVTIRTCGITTPTPSFRSVCKKIRLLGAT

SEQ ID NO: 8 - protein, 12 aa, synthetic construct
RTSSLPGEKEGS

SEQ ID NO: 9 - protein, 99 aa, synthetic construct
MEPLNPRQINNIKTTLEVLCFRVTNTLDPATDSTRGSRSTQQTRRPSSTTRPTTSSSRPETTRTSSTTTPTPSSRSGSKKIRLLGATRTSSLPGEKEGS

SEQ ID NO: 10 - protein, 87 aa, synthetic construct
MEPLNPRQINNIKTTLEVLCFRVTNTLDPATDSTRGSRSTQQTRRPSSTTRPTTSSSRPETTRTSSTTTPTPSSRSGSKKIRLLGAT

SEQ ID NO: 11 - protein, 100 aa, synthetic construct
LEPLNPRQINNIKTTLEVLCFRVTNTLDPATDSTRGSRSTQQTRRPSSTTRPTTSSSRPETTRTSSTTTPTPSSRSGSKKIRLLGATSGEQSSRPKRGFL

SEQ ID NO: 12 - protein, 609 aa, Adeno-associated virus 9
PLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - protein, 100 aa, synthetic construct
MEPRNPKPTSKSRTTAGVWCFLATSTSDPSTDSTRGSPSTRRMQRPSSTTRPTTSSSKRVTIRTCGITTPTPSFRSVCKKIRLLGATSGEQSSRPRRGFS

SEQ ID NO: 14 - protein, 709 aa, synthetic construct
MEPRNPKPTSKSRTTAGVWCFLATSTSDPSTDSTRGSPSTRRMQRPSSTTRPTTSSSKRVTIRTCGITTPTPSFRSVCKKIRLLGATSGEQSSRPRRGFSPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 15 - protein, 709 aa, synthetic construct
LEPLNPRQINNIKTTLEVLCFRVTNTLDPATDSTRGSRSTQQTRRPSSTTRPTTSSSRPETTRTSSTTTPTPSSRSGSKKIRLLGATSGEQSSRPKRGFLPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 119 aa, Adeno-associated virus 6
MEPRNPKPTSKSRTTAGVWCFLATSTSDPSTDSTRGSPSTRRMQRPSSTTRPTTSSSKRVTIRTCGITTPTPSFRSVCKKIRLLGATSGEQSSRPRRGFSNLLVWLRKVLRRLLERNVR

SEQ ID NO: 17 - protein, 119 aa, Adeno-associated virus 9
MEPLNPRQINNIKTTLEVLCFRVTNTLDPATDSTRGSRSTQQTRRPSSTTRPTTSSSRPETTRTSSTTTPTPSSRSGSKKIRLLGATSGEQSSRPKRGFLNLLVWLRKRLRRLLERRGL

SEQ ID NO: 18 - protein, 609 aa, Adeno-associated virus 6
PFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 19 - protein, 7 aa, synthetic construct
LGETTRP

SEQ ID NO: 20 - protein, 10 aa, synthetic construct
LALGETTRPA

(19) United States
(12) Patent Application Publication
(10) Pub. No.: US 2025/0304920 A1
(43) Pub. Date: Oct. 2, 2025

(54) Compositions and Methods for Enhanced Production of Adeno-Associated Virus

(71) Applicant: The Regents of the University of California, Oakland, CA (US)

(72) Inventors: David V. Schaffer, Oakland, CA (US); Hyuncheol Lee, Oakland, CA (US); Adam Joseph Schieferecke, Oakland, CA (US)

(21) Appl. No.: 18/863,226

(22) PCT Filed: May 9, 2023
(86) PCT No.: PCT/US2023/066774

§ 371 Date: Nov. 5, 2024

Related U.S. Application Data
(60) Provisional application No. 63/341,935, filed on May 13, 2022.
(60) Provisional application No. 63/433,054, filed on Dec. 16, 2022.

(51) Int. Cl. C12N 7/00; C07K 14/005; C12N 15/86

(52) U.S. Cl. CPC C12N 7/00; C07K 14/005; C12N 15/86; C12N 2750/14121; C12N 2750/14122; C12N 2750/14143; C12N 2750/14151

(57) ABSTRACT

The present disclosure provides variant membrane-associated accessory polypeptides (MAAP). The present disclosure provides recombinant AAV (rAAV) comprising a nucleotide sequence encoding a variant MAAP of the present disclosure. The present disclosure provides methods producing rAAV virions, using a variant MAAP of the present disclosure.

### CROSS-REFERENCE

This application claims the benefit of U.S. Provisional Patent Application No. 63/341,935, filed May 13, 2022, and U.S. Provisional Patent Application No. 63/433,054, filed Dec. 16, 2022, which applications are incorporated herein by reference in their entirety.

### INCORPORATION-BY-REFERENCE OF MATERIAL ELECTRONICALLY SUBMITTED

A Sequence Listing is provided herewith as a Sequence Listing XML, “BERK-467WO_SEQ_LIST” created on May 2, 2023 and having a size of 22,163 bytes. The contents of the Sequence Listing XML are incorporated by reference herein in their entirety.

### INTRODUCTION

Gene therapy is a therapeutic modality that involves the delivery of nucleic acid, such as DNA, to a cell, e.g., to treat a disease. Common delivery technologies include viral vectors, lipid delivery, and naked-DNA delivery, and while the latter two technologies boast low immune profiles, repeat administration ability, and lack of transgene size limit, the technologies are highly inefficient in vivo. Viral vectors are far more efficient and include a number of properties that make them advantageous. A currently used viral vector for in vivo delivery is Adeno-Associated Virus (AAV). AAV exhibits low immunogenicity and low random integration rate, making it one of the safest DNA delivery methods. DNA encoding a gene product to be delivered can be incorporated into the AAV genome, generating a recombinant AAV (rAAV).

AAVs are members of the Parvovirus family. The natural genome of AAV contains ˜4.7 kb of single-stranded DNA encodes up to ten known viral proteins. The rep gene encodes four protein products that facilitate genomic replication and play essential roles in loading nascent ssDNA genomes into assembled capsids. The cap region, which lies 3′ of the rep gene, encodes the protein products VP1, VP2, and VP3, which are structural proteins that assemble to form the capsid, the assembly activating protein (AAP), which targets VP proteins to the nucleus and is involved in capsid assembly, and the recently discovered membrane-associated accessory protein (MAAP). Nucleic acid encoding a gene product to be delivered can be inserted into the AAV genome in place of viral genes, generating a recombinant AAV (rAAV).

As clinical trials using, and Food & Drug Administration-approved products including, AAV have increased in number, and the understanding of AAV's natural replication cycle has deepened, technologies for rAAV manufacturing have also been developed. Various platforms, such as engineered HeLa cell systems or baculovirus production systems, are available. The most commonly used method for rAAV manufacturing involves plasmid DNA transfection into human embryonic kidney 293 (HEK293) cells. Despite the existence of methods for rAAV manufacturing, producing rAAV in sufficient quantities to meet increasing clinical demand remains a challenge.

There is a need in the art for methods for producing rAAV virions.

### SUMMARY

The present disclosure provides variant membrane-associated accessory polypeptides (MAAP). The present disclosure provides recombinant AAV (rAAV) comprising a nucleotide sequence encoding a variant MAAP of the present disclosure. The present disclosure provides methods producing rAAV virions, using a variant MAAP of the present disclosure.

### BRIEF DESCRIPTION OF THE DRAWINGS

FIG. 1A-1F depict a directed evolution method for identification of membrane associated accessory protein (MAAP) variants that confer increased packaging levels for AAV.

FIG. 2A-2D depict head-to-head packaging comparison of a green fluorescent protein (GFP) transgene into an AAV2 capsid in HEK293 cells stably expressing selected leading MAAP variants.

FIG. 3 provides a Table that presents amino acid sequences of MAAP variants (SEQ ID NOs: 1-5, respectively).

FIG. 4A-4F provide amino acid sequences of MAAP variants (FIG. 4A-4D, SEQ ID NOs: 6, 9, 14, and 15, respectively) and wild-type MAAP (FIG. 4E and FIG. 4F, SEQ ID NOs: 16-17, respectively).

FIG. 5A-5B depict head-to-head packaging comparison of a GFP transgene into an AAV9 capsid in HEK293 cells stably expressing selected MAAP variants.

FIG. 6A-6B depict head-to-head packaging comparison of a GFP transgene into an AAV6 capsid in HEK293 cells stably expressing selected MAAP variants.

FIG. 7A-7B depict head-to-head infectious titer comparison of AAV6-mediated delivery of a GFP transgene that was packaged into an AAV6 capsid in HEK293 cells stably expressing selected MAAP variants.

FIG. 8A-8B depict a method for inactivation of endogenous MAAP expression from an AAV2 genome that does not affect the VP1 open reading frame.

### DEFINITIONS

“AAV” is an abbreviation for adeno-associated virus, and may be used to refer to the virus itself or derivatives thereof. The term covers all subtypes and both naturally occurring and recombinant forms, except where required otherwise. The term encompasses AAV with variant capsids. The abbreviation “rAAV” refers to recombinant adeno-associated virus, also referred to as a recombinant AAV vector (or “rAAV vector”). The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, and ovine AAV. See, e.g., Mori et al. (2004) Virology 330:375. The term “AAV” also includes chimeric AAV. “Primate AAV” refers to AAV isolated from a primate, “non-primate AAV” refers to AAV isolated from a non-primate mammal, “bovine AAV” refers to AAV isolated from a bovine mammal (e.g., a cow), etc.

An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids.

An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e. a polynucleotide other than a wild-type AAV genome, such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

“Packaging” refers to a series of intracellular events that result in the assembly and encapsidation of an AAV particle.

AAV “rep” and “cap” genes refer to polynucleotide sequences encoding replication and encapsidation proteins of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

An “infectious” virus or viral particle is one that comprises a polynucleotide component which it is capable of delivering into a cell for which the viral species is tropic. The term does not necessarily imply any replication capacity of the virus. As used herein, an “infectious” virus or viral particle is one that can access a target cell, can infect a target cell, and can express a heterologous nucleic acid in a target cell. Thus, “infectivity” refers to the ability of a viral particle to access a target cell, infect a target cell, and express a heterologous nucleic acid in a target cell. Infectivity can refer to in vitro infectivity or in vivo infectivity. Assays for counting infectious viral particles are described elsewhere in this disclosure and in the art. Viral infectivity can be expressed as the ratio of infectious viral particles to total viral particles. Total viral particles can be expressed as the number of viral genome (vg) copies. The ability of a viral particle to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA). Viral infectivity can be expressed as the ratio of infectious viral particles to total viral particles. Methods of determining the ratio of infectious viral particle to total viral particle are known in the art. See, e.g., Grainger et al. (2005) Mol. Ther. 11:S337 (describing a TCID50 infectious titer assay); and Zolotukhin et al. (1999) Gene Ther. 6:973.

A “replication-competent” virus (e.g. a replication-competent AAV) refers to a phenotypically wild-type virus that is infectious, and is also capable of being replicated in an infected cell (i.e. in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector. In many embodiments, rAAV vector preparations as described herein are those which contain few if any replication competent AAV (rcAAV, also referred to as RCA) (e.g., less than about 1 rcAAV per 102 rAAV particles, less than about 1 rcAAV per 104 rAAV particles, less than about 1 rcAAV per 108 rAAV particles, less than about 1 rcAAV per 1012 rAAV particles, or no rcAAV).

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the present disclosure that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally-occurring, wild-type AAV, and the encoded heterologous gene product is a gene product not normally encoded by a naturally-occurring, wild-type AAV. As another example, in connection with a CRISPR-Cas fusion polypeptide comprising a CRISPR-Cas effector polypeptide and a heterologous fusion partner, a heterologous fusion partner is a polypeptide that is not normally linked to the CRISPR-Cas effector polypeptide in nature.

The terms “genetic alteration” and “genetic modification” (and grammatical variants thereof), are used interchangeably herein to refer to a process wherein a genetic element (e.g., a polynucleotide) is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a DNA or RNA virus or viral vector. Generally, the genetic element is introduced into a chromosome or mini-chromosome in the cell; but any alteration that changes the phenotype and/or genotype of the cell and its progeny is included in this term.

A cell is said to be “stably” altered, transduced, genetically modified, or transformed with a genetic sequence if the sequence is available to perform its function during extended culture of the cell in vitro. Generally, such a cell is “heritably” altered (genetically modified) in that a genetic alteration is introduced which is also inheritable by progeny of the altered cell.

The terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides such as anti-angiogenic polypeptides, neuroprotective polypeptides, and the like, when discussed in the context of delivering a gene product to a mammalian subject, and compositions therefor, refer to the respective intact polypeptide, or any fragment or genetically engineered derivative thereof, which retains the desired biochemical function of the intact protein. Similarly, references to nucleic acids encoding anti-angiogenic polypeptides, nucleic acids encoding neuroprotective polypeptides, and other such nucleic acids for use in delivery of a gene product to a mammalian subject (which may be referred to as “transgenes” to be delivered to a recipient cell), include polynucleotides encoding the intact polypeptide or any fragment or genetically engineered derivative possessing the desired biochemical function.

An “isolated” plasmid, nucleic acid, vector, virus, virion, host cell, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this disclosure are increasingly more isolated. An isolated nucleic acid, vector, virus, host cell, or other substance is in some embodiments purified, e.g., from about 80% to about 90% pure, at least about 90% pure, at least about 95% pure, at least about 98% pure, or at least about 99%, or more, pure.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and arc also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a variant MAAP polypeptide” includes a plurality of such polypeptides and reference to “the recombinant AAV (rAAV) virion” includes reference to one or more rAAV virions and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides variant membrane-associated accessory polypeptides (MAAP). The present disclosure provides recombinant AAV (rAAV) comprising a nucleotide sequence encoding a variant MAAP of the present disclosure. The present disclosure provides methods producing rAAV virions, using a variant MAAP of the present disclosure.

### Variant MAAPS

The present disclosure provides variant adeno-associated virus (AAV) membrane-associated accessory polypeptides (MAAP).

In some cases, a variant MAAP of the present disclosure comprises an amino acid sequence having at least 50%, at least 60%, at least 70%, at least 75%, at least 80%, at least 85%, at least 90%, at least 95%, at least 98%, at least 99%, or 100%, amino acid sequence identity to any one of the amino acid sequences depicted in FIG. 3.

In some cases, a variant MAAP of the present disclosure comprises an amino acid sequence having at least 50%, at least 60%, at least 70%, at least 75%, at least 80%, at least 85%, at least 90%, at least 95%, at least 98%, at least 99%, or 100%, amino acid sequence identity to the following amino acid sequence: MAHHHQGPQRGIRATAGVLCFLGTSTSDPSTDTSRESRSTRQTPRPLSMSKPTTGSPTADNTRTS SATSPTRSLGAPKGCYVFRGQTRTSSLPGEKEGS* (SL01; SEQ ID NO:1), where the variant MAAP has a length of from 100 amino acids to 104 amino acids. Such a variant MAAP may be referred to herein as a “SL01 MAAP” or simply “SL01.” In some cases, a SL01 MAAP has a length of 104 amino acids.

In some cases, a variant MAAP of the present disclosure comprises an amino acid sequence having at least 50%, at least 60%, at least 70%, at least 75%, at least 80%, at least 85%, at least 90%, at least 95%, at least 98%, at least 99%, or 100%, amino acid sequence identity to the following amino acid sequence: MAHHHQSPQSGIRTTAGVLCFLGTSTGPFSGLDKGEPVIEADAAALDHDKAYDRQLDGGDNLY LKYNHADAEFQERLKEDTSCGGNLGRAVFQANKRVPEPLGLVEKPVKTAPGKKRPG (SL08; SEQ ID NO:2), where the variant MAAP has a length of from 120 amino acids to 125 amino acids. Such a variant MAAP may be referred to herein as a “SL08 MAAP” or simply “SL08.” In some cases, a SL08 MAAP has a length of 125 amino acids.

In some cases, a variant MAAP of the present disclosure comprises an amino acid sequence having at least 50%, at least 60%, at least 70%, at least 75%, at least 80%, at least 85%, at least 90%, at least 95%, at least 98%, at least 99%, or 100%, amino acid sequence identity to the following amino acid sequence: MAHHHPSPQSGIRTTAGVSCFLGTSTPDPSTDSTRECRSTRQTPRPSSTTKPTTTARQRRQPVPQV QPRRRGVSGAP (SL30; SEQ ID NO:3), where the variant MAAP has a length of from 78 amino acids to 82 amino acids. Such a variant MAAP may be referred to herein as a “SL30 MAAP” or simply “SL30.” In some cases, a SL30 MAAP has a length of 82 amino acids.

In some cases, a variant MAAP of the present disclosure comprises an amino acid sequence having at least 50%, at least 60%, at least 70%, at least 75%, at least 80%, at least 85%, at least 90%, at least 95%, at least 98%, at least 99%, or 100%, amino acid sequence identity to the following amino acid sequence: MAHHHQSPQGGIRTTAGVLCFLGTSTSDPSTDSTRESPSTRQTPRPSSSTKPTAGNLTVETTRISST TRRRGVSGAP* (SL31; SEQ ID NO:4), where the variant MAAP has a length of from 78 amino acids to 82 amino acids. Such a variant MAAP may be referred to herein as a “SL31 MAAP” or simply “SL31.” In some cases, a SL31 MAAP has a length of 82 amino acids.

In some cases, a variant MAAP of the present disclosure comprises an amino acid sequence having at least 50%, at least 60%, at least 70%, at least 75%, at least 80%, at least 85%, at least 90%, at least 95%, at least 98%, at least 99%, or 100%, amino acid sequence identity to the following amino acid sequence: MAHHHQSPQSGIRTTAGVLCFLGTSTSDPSTDSTRESRSTRQTPRPSSTTKPTTDSSTAETTRTSST TTPTRSFRSALKKIRHLGATSDEQSSGEKEGS* (SL35; SEQ ID NO:5), where the variant MAAP has a length of from 100 amino acids to 104 amino acids. Such a variant MAAP may be referred to herein as a “SL35 MAAP” or simply “SL35.” In some cases, a SL35 MAAP has a length of 104 amino acids.

In some cases, a variant MAAP of the present disclosure comprises an amino acid sequence having at least 50%, at least 60%, at least 70%, at least 75%, at least 80%, at least 85%, at least 90%, at least 95%, at least 98%, or at least 99%, amino acid sequence identity to: (a) the following SL01 MAAP amino acid sequence: MAHHHQGPQRGIRATAGVLCFLGTSTSDPSTDTSRESRSTRQTPRPLSMSKPTTGSPTADNTRTS SATSPTRSLGAPKGCYVFRGQTRTSSLPGEKEGS* (SL01; SEQ ID NO:1); or (b) the following SL35 MAAP amino acid sequence: MAHHHQSPQSGIRTTAGVLCFLGTSTSDPSTDSTRESRSTRQTPRPSSTTKPTTDSSTAETTRTSST TTPTRSFRSALKKIRHLGATSDEQSSGEKEGS* (SL35; SEQ ID NO:5), where amino acid 7 is G or S, amino acid 10 is R or S, amino acid 14 is A or T, amino acid 33 is T or S, amino acid 34 is S or T, amino acid 49 is L or S, amino acid 51 is M or T, amino acid 52 is S or T, amino acid 58 is G or D, amino acid 60 is P or S, amino acid 63 is D or E, amino acid 64 is N or T, amino acid 71 is A or T, and amino acid 73 is S or T; and where the variant MAAP has a length of from 100 amino acids to 104 amino acids.

In some cases, a variant MAAP of the present disclosure comprises an amino acid sequence having at least 50%, at least 60%, at least 70%, at least 75%, at least 80%, at least 85%, at least 90%, at least 95%, at least 98%, or at least 99%, amino acid sequence identity to: (a) the following SL30 MAAP amino acid sequence: MAHHHPSPQSGIRTTAGVSCFLGTSTPDPSTDSTRECRSTRQTPRPSSTTKPTTTARQRRQPVPQV QPRRRGVSGAP (SL30; SEQ ID NO:3); or (b) the following SL31 amino acid sequence: MAHHHQSPQGGIRTTAGVLCFLGTSTSDPSTDSTRESPSTRQTPRPSSSTKPTAGNLTVETTRISST TRRRGVSGAP* (SL31; SEQ ID NO:4), where the MAAP comprises an amino acid sequence having at least 75% amino acid sequence identity to the amino acid sequence of SL30 or SL31, and wherein amino acid 6 is P or Q, amino acid 10 is S or G, amino acid 19 is S or L, amino acid 38 is C or S, amino acid 39 is R or P, and amino acid 51 is T or S; and where the variant MAAP has a length of from 78 amino acids to 82 amino acids.

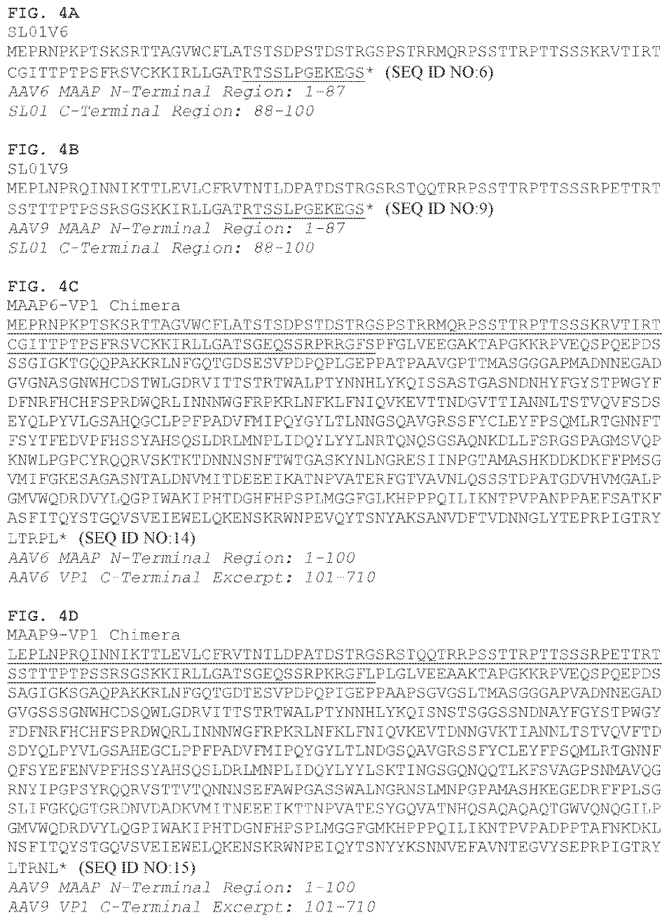

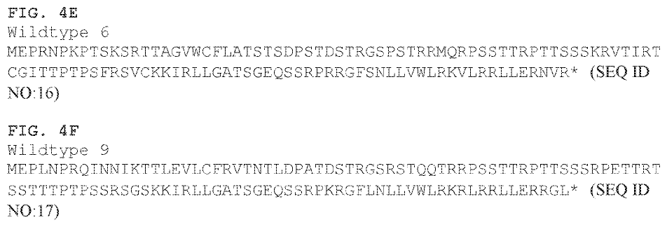

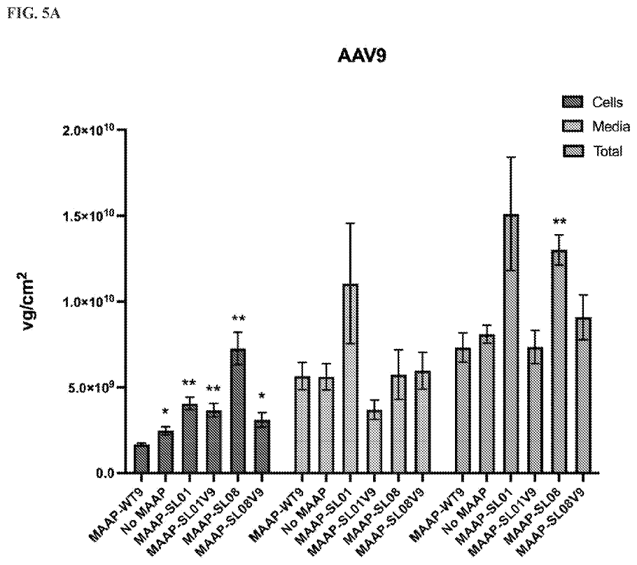

In some cases, a variant MAAP of the present disclosure comprises an amino acid sequence having at least 50%, at least 60%, at least 70%, at least 75%, at least 80%, at least 85%, at least 90%, at least 95%, at least 98%, at least 99%, or 100%, amino acid sequence identity to the following amino acid sequence: MEPRNPKPTSKSRTTAGVWCFLATSTSDPSTDSTRGSPSTRRMQRPSSTTRPTTSSSKRVTIRTCG ITTPTPSFRSVCKKIRLLGATRTSSLPGEKEGS (SEQ ID NO:6), which is also presented in FIG. 4A.

In some cases, a variant MAAP of the present disclosure comprises: a) an amino acid sequence having at least 50%, at least 60%, at least 70%, at least 75%, at least 80%, at least 85%, at least 90%, at least 95%, at least 98%, at least 99%, or 100%, amino acid sequence identity to: MEPRNPKPTSKSRTTAGVWCFLATSTSDPSTDSTRGSPSTRRMQRPSSTTRPTTSSSKRVTIRTCG ITTPTPSFRSVCKKIRLLGAT (SEQ ID NO:7); and b) the amino acid sequence RTSSLPGEKEGS (SEQ ID NO:8).

In some cases, a variant MAAP of the present disclosure comprises an amino acid sequence having at least 50%, at least 60%, at least 70%, at least 75%, at least 80%, at least 85%, at least 90%, at least 95%, at least 98%, at least 99%, or 100%, amino acid sequence identity to the following amino acid sequence: MEPLNPRQINNIKTTLEVLCFRVTNTLDPATDSTRGSRSTQQTRRPSSTTRPTTSSSRPETTRTSST TTPTPSSRSGSKKIRLLGATRTSSLPGEKEGS (SEQ ID NO:9), which is also presented in FIG. 4B. In some cases, a variant MAAP of the present disclosure comprises: a) an amino acid sequence having at least 50%, at least 60%, at least 70%, at least 75%, at least 80%, at least 85%, at least 90%, at least 95%, at least 98%, at least 99%, or 100%, amino acid sequence identity to: MEPLNPRQINNIKTTLEVLCFRVTNTLDPATDSTRGSRSTQQTRRPSSTTRPTTSSSRPETTRTSST TTPTPSSRSGSKKIRLLGAT (SEQ ID NO:10); and b) the amino acid sequence RTSSLPGEKEGS (SEQ ID NO:8).

In some cases, a variant MAAP of the present disclosure is a fusion polypeptide comprising: a) a MAAP polypeptide (which may be a variant MAAP polypeptide as described above); and b) an AAV VP1 polypeptide. Non-limiting examples of such fusion polypeptides are provided in FIG. 4C and FIG. 4D. The fusion polypeptide depicted in FIG. 4D is referred to as “MAPP9-VP1 Chimera” or “MAAP-SL08V9”. For example, in some cases, a fusion polypeptide of the present disclosure comprises: a) a MAAP comprising an amino acid sequence having at least 50%, at least 60%, at least 70%, at least 75%, at least 80%, at least 85%, at least 90%, at least 95%, at least 98%, at least 99%, or 100%, amino acid sequence identity to the following amino acid sequence: LEPLNPRQINNIKTTLEVLCFRVTNTLDPATDSTRGSRSTQQTRRPSSTTRPTTSSSRPETTRTSSTT TPTPSSRSGSKKIRLLGATSGEQSSRPKRGFL (SEQ ID NO:11); and b) an AAV VP1 polypeptide comprising an amino acid sequence having at least 50%, at least 60%, at least 70%, at least 75%, at least 80%, at least 85%, at least 90%, at least 95%, at least 98%, at least 99%, or 100%, amino acid sequence identity to the following amino acid sequence:

(SEQ ID NO: 12)
PLGLVEEAAKTAPGKKRPVEQSPQEPDSSAGIGKSGAQPAKKRLNFGQTG
DTESVPDPQPIGEPPAAPSGVGSLTMASGGGAPVADNNEGADGVGSSSGN
WHCDSQWLGDRVITTSTRTWALPTYNNHLYKQISNSTSGGSSNDNAYFGY
STPWGYFDFNRFHCHFSPRDWQRLINNNWGFRPKRLNFKLFNIQVKEVTD
NNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYA
HSQSLDRLMNPLIDQYLYYLSKTINGSGQNQQTLKFSVAGPSNMAVQGRN
YIPGPSYRQQRVSTTVTQNNNSEFAWPGASSWALNGRNSLMNPGPAMASH
KEGEDRFFPLSGSLIFGKQGTGRDNVDADKVMITNEEEIKTTNPVATESY
GQVATNHQSAQAQAQTGWVQNQGILPGMVWQDRDVYLQGPIWAKIPHTDG
NFHPSPLMGGFGMKHPPPQILIKNTPVPADPPTAFNKDKLNSFITQYSTG
QVSVEIEWELQKENSKRWNPEIQYTSNYYKSNNVEFAVNTEGVYSEPRPI
GTRYLTRNL.

As another example, in some cases, a fusion polypeptide of the present disclosure comprises: a) a MAAP comprising an amino acid sequence having at least 50%, at least 60%, at least 70%, at least 75%, at least 80%, at least 85%, at least 90%, at least 95%, at least 98%, at least 99%, or 100%, amino acid sequence identity to the following amino acid sequence: MEPRNPKPTSKSRTTAGVWCFLATSTSDPSTDSTRGSPSTRRMQRPSSTTRPTTSSSKRVTIRTCG ITTPTPSFRSVCKKIRLLGATSGEQSSRPRRGFS (SEQ ID NO:13); and b) an AAV VP1 polypeptide comprising an amino acid sequence having at least 50%, at least 60%, at least 70%, at least 75%, at least 80%, at least 85%, at least 90%, at least 95%, at least 98%, at least 99%, or 100%, amino acid sequence identity to the following amino acid sequence:

(SEQ ID NO: 12)
PLGLVEEAAKTAPGKKRPVEQSPQEPDSSAGIGKSGAQPAKKRLNFGQTG
DTESVPDPQPIGEPPAAPSGVGSLTMASGGGAPVADNNEGADGVGSSSGN
WHCDSQWLGDRVITTSTRTWALPTYNNHLYKQISNSTSGGSSNDNAYFGY
STPWGYFDFNRFHCHFSPRDWQRLINNNWGFRPKRLNFKLFNIQVKEVTD
NNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYA
HSQSLDRLMNPLIDQYLYYLSKTINGSGQNQQTLKFSVAGPSNMAVQGRN
YIPGPSYRQQRVSTTVTQNNNSEFAWPGASSWALNGRNSLMNPGPAMASH
KEGEDRFFPLSGSLIFGKQGTGRDNVDADKVMITNEEEIKTTNPVATESY
GQVATNHQSAQAQAQTGWVQNQGILPGMVWQDRDVYLQGPIWAKIPHTDG
NFHPSPLMGGFGMKHPPPQILIKNTPVPADPPTAFNKDKLNSFITQYSTG
QVSVEIEWELQKENSKRWNPEIQYTSNYYKSNNVEFAVNTEGVYSEPRPI
GTRYLTRNL.

A variant MAAP of the present disclosure, when present in a producer cell, provides for increased production of rAAV virions from the producer cell. For example, a variant MAAP of the present disclosure, when present in a producer cell, provides a level of production of rAAV virions that is at least 10%, at least 15%, at least 20%, at least 25%, at least 30%, at least 40%, at least 50%, at least 60%, at least 70%, at least 80%, at least 90%, at least 100% (or two-fold), at least 2.5-fold, at least 5-fold, at least 10-fold, or more than 10-fold, higher than the level of production when the producer cell comprises a nucleotide sequence encoding a wild-type MAAP. In some cases, at least 50%, at least 55%, at least 60%, at least 70%, at least 80%, at least 90%, or more than 90%, of the rAAV virions are secreted into the culture medium in which the producer cell is cultured.

Suitable producer cells include, e.g., mammalian cell lines. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HEK 293T cells, HLHepG2 cells, and the like.

### Nucleic Acids

The present disclosure provides a nucleic acid comprising a nucleotide sequence encoding a variant MAAP of the present disclosure. In some cases, the nucleotide sequence is operably linked to a transcriptional control element, e.g., a promoter. In some cases, the promoter is a cell type-specific promoter or a tissue-specific promoter. In some cases, the promoter is a regulatable promoter (e.g., an inducible promoter). In some cases, the promoter is a constitutive promoter.

In some cases, the promoter is a tissue-specific promoter. “Tissue-specific promoters” are known to the art and include, but are not limited to, neuron-specific promoters, muscle-specific promoters, liver-specific promoters, skeletal muscle-specific promoters, adipocyte-specific promoters, and cardiomyocyte-specific promoters. “Neuron-specific promoters” are known to the art and include, but are not limited to, the synapsin 1 (SYN) promoter, the calcium/calmodulin-dependent protein kinase IT promoter, the tubulin alpha 1 promoter, the neuron-specific enolase promoter, and the platelet-derived growth factor beta chain promoter. Liver-specific promoters” are known to the art and include, but are not limited to, the α1-microglobulin/bikunin enhancer/thyroid hormone-binding globulin promoter, the human albumin (hALB) promoter, the thyroid hormone-binding globulin promoter, thyroxin binding globulin promoter, the α1-anti-trypsin promoter, the bovine albumin (bAlb) promoter, the murine albumin (mAlb) promoter, the human α1-anti-trypsin (hAAT) promoter, the ApoEhAAT promoter composed of the ApoE enhancer and the hAAT promoter, the transthyretin (TTR) promoter, the liver fatty acid binding protein promoter, the hepatitis B virus (HBV) promoter, the DCI 72 promoter consisting of the hAAT promoter and the α1-microglobulin enhancer, the DC 190 promoter containing the human albumin promoter and the prothrombin enhancer, and other natural and synthetic liver-specific promoters. “Muscle-specific promoters” are known to the art and include, but are not limited to, the MHCK7 promoter, the muscle creatine kinase (MCK) promoter/enhancer, the slow isoform of troponin 1 (THIS) promotor, the MYODI promoter, the MYLK2 promoter, the SPc5-12 promoter, the desmin (Des) promoter, the unc45b promoter, and other natural and synthetic muscle-specific promoters. “Skeletal muscle-specific promoters” are known to the art and include, but are not limited to, the HSA promoter, the human α-skeletal actin promoter. “Heart-specific promoters” (cardiomyocyte-specific promoters) are known to the art and include, but art not limited to, the MYH6 promoter, the TNN13 promoter, the cardiac troponin C (cTnC) promoter, the alpha-myosin heavy chain (α-MHC) promoter, myosin light chain 2 (MLC-2), and the MYBPC3 promoter.

In some cases, the promoter is a constitutive promoter. A constitutive promoter can be used to express genes in a wide range of cells and tissues, including, but not limited to, the liver, kidney, skeletal muscle, cardiac muscle, smooth muscle, diaphragm muscle, brain, spinal coni, endothelial cells, intestinal cells, pulmonary cells (e.g., smooth muscle or epithelium), peritoneal epithelial cells, and fibroblasts. Suitable constitutive promoters include, but are not limited to, a cytomegalovirus (CMV) major immediate-early enhancer/chicken beta-actin promoter, a CMV major immediate-early promoter, an Elongation Factor I-a (EPI-a) promoter, a simian vacuolating virus 40 (SV40) promoter, an AmpR promoter, a PγK promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, a β-kin promoter, a murine phosphoglycerate kinase (mPGK) or human PGK (hPGK) promoter, a ROSA promoter, human Ubiquitin B promoter, a Rous sarcoma virus promoter, or any other natural or synthetic constitutive promoter.

In some cases, the promoter is an AAV promoter, e.g., an AAV p40 promoter.

In some cases, the nucleic acid is present in a recombinant vector, e.g., a recombinant viral vector.

### Host Cells

The present disclosure provides a modified eukaryotic cell (e.g., genetically modified eukaryotic cells), where the modified eukaryotic cell comprises a nucleic acid of the present disclosure, i.e., comprises a nucleic acid comprising a nucleotide sequence encoding a variant MAAP of the present disclosure. The modified eukaryotic cell is in vitro. In some cases, the nucleic acid comprising a nucleotide sequence encoding a variant MAAP is integrated into the genomic DNA of the eukaryotic cell. In some cases, the modified in vitro eukaryotic cell is one that grows in culture as a single-cell suspension. In some cases, the modified in vitro eukaryotic cell is a modified mammalian cell line. A modified eukaryotic cell of the present disclosure is useful for producing a recombinant AAV (rAAV) virion.

Suitable cells that can be modified to produce a modified eukaryotic cell of the present disclosure include, e.g., mammalian cell lines. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HEK 293T cells, HLHepG2 cells, and the like.

Suitable cells that can be modified to produce a modified eukaryotic cell of the present disclosure include, e.g., Spodoptera frugiperda drosophila cell lines, or mosquito cell lines (e.g., Aedes albopictus-derived cell lines). In some cases, the insect cell is susceptible to baculovirus infection (e.g., Se301, SeIZD2109, SeUCR1, Sf9, Sf900+, Sf21, BTI-TN-5B1-4, MG-1, Tn368, HzAm1, Ha2302, and Hz2E5). Such cells are suitable for producing rAAV using baculovirus expression vectors (BEVs). See, e.g., U.S. Patent Publication No. 2020/0123572.

A genetically modified eukaryotic cell of the present disclosure can function as “producer” cell for AAV vector replication and packaging. Such a producer cell generally comprises or is modified to comprise several different types of components for AAV (including rAAV) production. In some cases, a genetically modified mammalian cell of the present disclosure can comprise an AAV genome or a recombinant adeno-associated viral (rAAV) vector genome (or “rAAV pro-vector”) that can be replicated and packaged into particles by the host packaging cell. The rAAV pro-vector comprises a heterologous nucleic acid (or “transgene”), where the heterologous nucleic acid can be flanked by two AAV inverted terminal repeats (ITRs) which comprise sequences that are recognized during excision, replication and packaging of the AAV vector, as well as during integration of the vector into a host cell genome.

A genetically modified eukaryotic cell of the present disclosure can comprise a helper virus that can provide helper functions for AAV replication, or a helper recombinant vector that comprises a nucleotide sequence encoding helper functions. Although adenovirus is commonly employed, other helper viruses can also be used as is known in the art. Alternatively, the requisite helper virus functions can be isolated genetically from a helper virus and the encoding genes can be used to provide helper virus functions in trans. The AAV vector elements and the helper virus (or helper virus functions) can be introduced into the host cell either simultaneously or sequentially in any order.

A genetically modified eukaryotic cell of the present disclosure can comprise “AAV packaging genes” such as AAV rep and cap genes that provide replication and encapsidation proteins, respectively. Several different versions of AAV packaging genes can be provided (including rep-cap cassettes and separate rep and/or cap cassettes in which the rep and/or cap genes can be left under the control of the native promoters or operably linked to heterologous promoters. Such AAV packaging genes can be introduced either transiently or stably into the host packaging cell (e.g., a genetically modified eukaryotic cell of the present disclosure), as is known in the art.

In some cases, a genetically modified eukaryotic cell of the present disclosure is further genetically modified with one or more nucleic acids comprising nucleotide sequences encoding one or more of: AAV rep proteins; and AAV cap protein. In some cases, such nucleic acids encoding helper functions are integrated into the genome of a genetically modified mammalian cell of the present disclosure.

To produce an AAV virion (which may be an rAAV virion), a genetically modified eukaryotic cell of the present disclosure can comprise: a) a nucleic acid comprising a nucleotide sequence encoding a variant MAAP; b) a nucleic acid comprising a nucleotide sequence encoding an AAV capsid; and c) an rAAV comprising: i) AAV ITRs; and ii) a heterologous nucleic acid comprising a nucleotide sequence encoding one or more heterologous gene products). The modified eukaryotic cell can be modified with a helper virus or one or more recombinant expression vectors comprising nucleotide sequences encoding helper functions. Thus, in some cases, a method of the present disclosure comprises: A) culturing a genetically modified eukaryotic cell of the present disclosure in vitro in a liquid culture medium, where the genetically modified mammalian cell comprises: a) a nucleic acid comprising a nucleotide sequence encoding a variant MAAP; b) a nucleic acid comprising a nucleotide sequence encoding an AAV capsid; and c) an rAAV comprising: i) AAV ITRs; and ii) a heterologous nucleic acid comprising a nucleotide sequence encoding one or more heterologous gene products; B) introducing into the genetically modified mammalian cell a nucleic acid comprising a nucleotide sequence encoding helper functions, such that the genetically modified mammalian cell produces an AAV virion or an rAAV virion; and C) harvesting the AAV virion from the culture medium. Suitable liquid culture media include any culture medium that provides for growth and/or viability of a mammalian cell or an insect cell in in vitro culture.

In some cases, the harvested AAV virions (e.g., rAAV virions) are purified. In some cases, the AAV virions (e.g., rAAV virions) are purified to at least 80%, at least 90%, at least 95%, at least 98%, at least 99%, or greater than 99%, purity.

In some cases, the cap gene product is a variant cap polypeptide. In some cases, the variant AAV capsid protein comprises from 1 to about 10 amino acid differences (e.g., amino acid substitutions and/or amino acid insertions and/or amino acid deletions) compared to a wild-type AAV capsid protein. The amino acid difference(s) can be located in a solvent accessible site in the capsid, e.g., a solvent-accessible loop. For example, the amino acid substitution(s) can be located in a GH loop in the AAV capsid protein. As one non-limiting example, the variant capsid protein can comprise an amino acid substitution at amino acid 451 of AAV6 capsid, or the corresponding position in another AAV serotype. As another non-limiting example, the variant capsid protein can comprise an amino acid substitution at amino acid 532 of AAV6 capsid, or the corresponding position in another AAV serotype. In some cases, the variant capsid comprises an insertion of a peptide comprising LGETTRP (SEQ ID NO:19), where the insertion site is between amino acids corresponding to 570 and 611 of VP1 of AAV2, or the corresponding position in the capsid protein of another AAV serotype. In some cases, the variant capsid comprises an insertion of a peptide comprising LALGETTRPA (SEQ ID NO:20), where the insertion site is between amino acids corresponding to 570 and 611 of VP1 of AAV2, or the corresponding position in the capsid protein of another AAV serotype. See, e.g., U.S. Pat. Nos. 9,193,956; 8,663,624; and 9,457,103.

### RAAV Encoding Heterologous Gene Product(s)

In some cases, a modified eukaryotic cell of the present disclosure comprises a recombinant AAV (rAAV) comprising: (i) inverted terminal repeats; and (ii) a nucleic acid comprising a nucleotide sequence(s) encoding one or more heterologous gene products. Heterologous gene products can be polypeptides or nucleic acids, or a combination of polypeptides and nucleic acids.

Suitable heterologous gene products include polypeptides, where suitable polypeptides include, but are not limited to, a neuroprotective polypeptide, an anti-angiogenic polypeptide, a growth factor, a polypeptide that provides for enhanced function of a cell, a CRISPR-Cas effector polypeptide, and the like. Suitable heterologous gene products include: a) a type II CRISPR-Cas effector polypeptide, b a type II CRISPR-Cas effector polypeptide; c) a type V CRISPR-Cas effector polypeptide; d) a type VI CRISPR-Cas effector polypeptide; e) an enzymatically inactive CRISPR-Cas effector polypeptide; f) a nickase CRISPR-Cas effector polypeptide; g) a fusion polypeptide comprising: (i) a CRISPR-Cas effector polypeptide; and (ii) a heterologous fusion partner; and h) a CRISPR-Cas effector polypeptide and a guide RNA (e.g., a single-molecule guide RNA (a “single-guide” RNA). Suitable heterologous gene products include interfering RNAs. Suitable heterologous gene products include siRNAs. Suitable heterologous gene products include microRNAs. Suitable heterologous gene products include aptamers. Suitable heterologous gene products include fluorescent proteins (e.g., green fluorescent protein (GFP); cyan fluorescent protein; yellow fluorescent protein; red fluorescent protein; and the like).

As noted above, a heterologous gene product can be a Clustered Regularly Interspaced Short Palindromic Repeats (CRISPR)-associated (CRISPR-Cas) effector polypeptide. In some cases, the CRISPR-Cas effector polypeptide is a type II CRISPR-Cas effector polypeptide. In some cases, the type II CRISPR-Cas effector polypeptide is a Cas9 polypeptide. In some cases, the CRISPR-Cas effector polypeptide is a type V CRISPR-Cas effector polypeptide, e.g., a Cas12a, a Cas12b, a Cas12c, a Cas12d, or a Cas12e polypeptide. In some cases, the CRISPR-Cas effector polypeptide is a type VI CRISPR-Cas effector polypeptide, e.g., a Cas13a polypeptide, a Cas13b polypeptide, a Cas13c polypeptide, or a Cas13d polypeptide. In some cases, the CRISPR-Cas effector polypeptide is a Cas14 polypeptide. In some cases, the CRISPR-Cas effector polypeptide is a Cas14a polypeptide, a Cas14b polypeptide, or a Cas14c polypeptide. In some cases, the CRISPR-Cas effector polypeptide is a Cas7-11 polypeptide; see, e.g., Özcan et al. (2021) Nature 597:720. In some cases, the CRISPR-Cas effector polypeptide is a CRISPRi polypeptide; see, e.g., Qi et al. (2013) Cell 152:1173; and Jensen et al. (2021) Genome Research doi: 10.1101/gr.275607.121. In some cases, the CRISPR-Cas effector polypeptide is a CRISPRa polypeptide; see, e.g., Jensen et al. (2021) Genome Research doi: 10.1101/gr.275607.121; and Breinig et al. (2019) Nature Methods 16:51. In some cases, the CRISPR-Cas effector polypeptide is a CRISPRoff polypeptide. See, e.g., Nuñez et al. (2021) Cell 184:2503. In some cases, the CRISPR-Cas effector polypeptide is a nickase. In some cases, the CRISPR-Cas effector polypeptide exhibits reduced catalytic activity compared to a wild-type CRISPR-Cas effector polypeptide.

In some cases, the CRISPR-Cas effector polypeptide is a fusion polypeptide comprising: i) a CRISPR-Cas effector polypeptide; and ii) one or more heterologous fusion partners (also referred to as “heterologous polypeptides”), where the heterologous polypeptide is an effector polypeptide. Exemplary effector polypeptides include, e.g., polypeptides that can cleave RNA (e.g., a PIN endonuclease, an NYN domain, an SMR domain from SOT1, or an RNase domain from a Staphylococcal nuclease); polypeptides that can affect RNA stability (e.g., tristetraprolin (TTP) or domains from UPF1, EXOSC5, and STAU1); polypeptides that can modify a nucleotide or ribonucleotide (e.g., a cytidine deaminase, PPR protein, adenosine deaminase, an adenosine deaminase acting on RNA (ADAR) family protein, or an APOBEC family protein); polypeptides that can activate translation (e.g., eIF4E and other translation initiation factors, a domain of the yeast poly(A)-binding protein or GLD2), those that can repress translation (e.g., Pumilio or FBF PUF proteins, deadenylases, CAF1, Argonaute proteins); polypeptides that can methylate RNA (e.g., domains from m6A methyltransferase factors such as METTL14, METTL3, or WTAP); polypeptides that can demethylate RNA (e.g., human alkylation repair homolog 5 or Alkbh5); polypeptides that can affect splicing (e.g., the RS-rich domain of SRSF1, the Gly-rich domain of hnRNP A1, the alanine-rich motif of RBM4, or the proline-rich motif of DAZAP1); polypeptides that can enable affinity purification or immunoprecipitation (e.g., FLAG, hemagglutinin (HA), biotin, or HALO tags); and polypeptides that can enable proximity-based protein labeling and identification (e.g., a biotin ligase (such as BirA) or a peroxidase (such as APEX2) in order to biotinylate proteins that interact with the target RNA). Suitable heterologous polypeptides include splicing factors (e.g., RS domains); protein translation components (e.g., translation initiation, elongation, and/or release factors; e.g., eIF4G); RNA methylases; RNA editing enzymes (e.g., RNA deaminases, e.g., ADAR polypeptides, including A to I and/or C to U editing enzymes); helicases; RNA-binding proteins; and the like. In some cases, a heterologous polypeptide (a fusion partner) provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an endoplasmic reticulum (ER) retention signal, and the like).

A nucleotide sequence encoding a heterologous gene product in an rAAV virion of the present disclosure can be operably linked to a promoter. For example, a nucleotide sequence encoding a heterologous gene product in an rAAV virion can be operably linked to a constitutive promoter, a regulatable promoter, a tissue-specific promoter, or a cell type-specific promoter. In some cases, the promoter is an AAV promoter, e.g., an AAV p40 promoter.

Suitable nucleic acid gene products include interfering RNA, antisense RNA, ribozymes, CRISPR-Cas guide RNAs, and aptamers. Where the gene product is an interfering RNA (RNAi), suitable RNAi include RNAi that decrease the level of an angiogenic factor in a cell. For example, an RNAi can be a miRNA, an shRNA, or an siRNA that reduces the level of vascular endothelial growth factor (VEGF) in a cell.

Where the gene product is a polypeptide, exemplary polypeptides include, e.g., an interferon (e.g., IFN-γ, IFN-α, IFN-β, IFN-ω; IFN-τ); an insulin (e.g., Novolin, Humulin, Humalog, Lantus, Ultralente, etc.); an erythropoietin (“EPO”; e.g., Procrit®, Eprex®, or Epogen® (epoetin-α); Aranesp® (darbepoietin-α); NeoRecormon®, Epogin® (epoetin-β); and the like); an antibody (e.g., a monoclonal antibody) (e.g., Rituxan® (rituximab); Remicade® (infliximab); Herceptin® (trastuzumab); Humira™ (adalimumab); Xolair® (omalizumab); Bexxar® (tositumomab); Raptiva™ (efalizumab); Erbitux™ (cetuximab); and the like), including an antigen-binding fragment of a monoclonal antibody; a blood factor (e.g., Activase® (alteplase) tissue plasminogen activator; NovoSeven® (recombinant human factor VIIa); Factor VIIa; Factor VIII (e.g., Kogenate®); Factor IX; β-globin; hemoglobin; and the like); a colony stimulating factor (e.g., Neupogen® (filgrastim; G-CSF); Neulasta (pegfilgrastim); granulocyte colony stimulating factor (G-CSF), granulocyte-monocyte colony stimulating factor, macrophage colony stimulating factor, megakaryocyte colony stimulating factor; and the like); a growth hormone (e.g., a somatotropin, e.g., Genotropin®, Nutropin®, Norditropin®, Saizen®, Scrostim®, Humatrope®, etc.; a human growth hormone; and the like); an interleukin (e.g., IL-1; IL-2, including, e.g., Proleukin®; IL-3, IL-4, IL-5, IL-6, IL-7, IL-8, IL-9; etc.); a growth factor (e.g., Regranex® (beclapermin; PDGF); Fiblast® (trafermin; bFGF); Stemgen® (ancestim; stem cell factor); keratinocyte growth factor; an acidic fibroblast growth factor, a stem cell factor, a basic fibroblast growth factor, a hepatocyte growth factor; and the like); a soluble receptor (e.g., a TNF-α-binding soluble receptor such as Enbrel® (etanercept); a soluble VEGF receptor; a soluble interleukin receptor; a soluble γ/δ T cell receptor; and the like); an enzyme (e.g., α-glucosidase; Cerazyme® (imiglucarase; β-glucocerebrosidase, Ceredase® (alglucerase); an enzyme activator (e.g., tissue plasminogen activator); a chemokine (e.g., IP-10; Mig; Groα/IL-8, RANTES; MIP-1a; MIP-1B; MCP-1; PF-4; and the like); an angiogenic agent (e.g., vascular endothelial growth factor (VEGF); an anti-angiogenic agent (e.g., a soluble VEGF receptor); a protein vaccine; a neuroactive peptide such as bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, neurotensin, motilin, thyrotropin, neuropeptide Y, luteinizing hormone, calcitonin, insulin, glucagon, vasopressin, angiotensin II, thyrotropin-releasing hormone, vasoactive intestinal peptide, a sleep peptide, etc.; other proteins such as a thrombolytic agent, an atrial natriuretic peptide, bone morphogenic protein, thrombopoietin, relaxin, glial fibrillary acidic protein, follicle stimulating hormone, a human alpha-1 antitrypsin, a leukemia inhibitory factor, a transforming growth factor, an insulin-like growth factor, a luteinizing hormone, a macrophage activating factor, tumor necrosis factor, a neutrophil chemotactic factor, a nerve growth factor a tissue inhibitor of metalloproteinases; a vasoactive intestinal peptide, angiogenin, angiotropin, fibrin; hirudin; a leukemia inhibitory factor; an IL-1 receptor antagonist (e.g., Kineret® (anakinra)); an ion channel, e.g., cystic fibrosis transmembrane conductance regulator (CFTR); dystrophin; utrophin, a tumor suppressor; lysosomal enzyme acid α-glucosidase (GAA); and the like. Suitable nucleic acids also include those that encode a functional fragment of any of the aforementioned proteins; and nucleic acids that encode functional variants of any of the aforementioned proteins.

Where the gene product is a polypeptide, exemplary polypeptides include neuroprotective polypeptides and anti-angiogenic polypeptides. Suitable polypeptides include, but are not limited to, glial derived neurotrophic factor (GDNF), fibroblast growth factor 2 (FGF-2), neurturin, ciliary neurotrophic factor (CNTF), nerve growth factor (NGF; e.g., nerve growth factor-β), brain derived neurotrophic factor (BDNF), neurotrophin-3 (NT-3), neurotrophin-4 (NT-4), neurotrophin-6 (NT-6), epidermal growth factor (EGF), pigment epithelium derived factor (PEDF), a Wnt polypeptide, soluble Flt-1, angiostatin, endostatin, an anti-VEGF antibody, a soluble VEGFR, and a member of the hedgehog family (sonic hedgehog, indian hedgehog, and desert hedgehog, etc.).

The nucleotide sequence encoding the heterologous gene product(s) can be under the control of a promoter, e.g., a promoter that is functional in a eukaryotic cell. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Suitable reversible promoters, including reversible inducible promoters are known in the art. Suitable reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Suitable promoters include, but are not limited to, a CAG promoter (Miyazaki et al. (1989) Gene 79:269); a cytomegalovirus (CMV) promoter; a glutamate metabotropic receptor-6 (grm6) promoter (Cronin et al. (2014) EMBO Mol. Med. 6:1175); a Pleiades promoter (Portales-Casamar et al. (2010) Proc. Natl. Acad. Sci. USA 107:16589); a choline acetyltransferase (ChAT) promoter (Misawa et al. (1992) J. Biol. Chem. 267:20392); a vesicular glutamate transporter (V-glut) promoter (Zhang et al. (2011) Brain Res. 1377:1); a glutamic acid decarboxylase (GAD) promoter (Rasmussen et al. (2007) Brain Res. 1144:19; Ritter et al. (2016) J. Gene Med. 18:27); a cholecystokinin (CCK) promoter (Ritter et al. (2016) J. Gene Med. 18:27); a parvalbumin (PV) promoter; a somatostatin (SST) promoter; a neuropeptide Y (NPY) promoter; and a vasoactive intestinal peptide (VIP) promoter. Suitable promoters include, but are not limited to, a red cone opsin promoter, rhodopsin promoter, a rhodopsin kinase promoter, and a GluR promoter (e.g., a GluR6 promoter; also referred to as grm6). Suitable promoters include, but are not limited to, a vitelliform macular dystrophy 2 (VMD2) gene promoter, and an interphotoreceptor retinoid-binding protein (IRBP) gene promoter. Also suitable for use is an L7 promoter (Oberdick et al. (1990) Science 248:223), a thy-1 promoter, a recoverin promoter (Wiechmann and Howard (2003) Curr. Eye Res. 26:25); a calbindin promoter; and a beta-actin promoter. Suitable promoters include synthetic (non-naturally occurring) promoter/enhancer combinations. In some cases, the promoter is a retinal cell-specific promoter. In some cases, the promoter is a muscle cell-specific promoter. In some cases, the promoter is a neuron-specific promoter.

In some cases, the promoter is a human synapsin (hSyn) promoter, a human elongation factor 1-α (EF1α) promoter, a cytomegalovirus (CMV) promoter, a CMV early enhancer/chicken β actin (CAG) promoter, a synapsin-I promoter (e.g., a human synapsin-I promoter), a human synuclein 1 promoter, a human Thy1 promoter, a calcium/calmodulin-dependent kinase II alpha (CAMKIIα) promoter, a vesicular γ-amino butyric acid (VGAT) promoter, a glial fibrillary acidic protein (GFAP) promoter, a Pet1 promoter, a neuropeptide Y (NPY) promoter, a somatostatin (SST) promoter, an arginine vasopressin (AVP) promoter, or a hypocretin (Hert) promoter.

Suitable promoters include, e.g., a CamKII promoter, a human synapsin promoter, a Thy1 promoter, a glial fibrillary acid protein (GFAP) promoter (see, e.g., Lee et al. (2008) Glia 56:481), a vesicular gamma amino butyric acid transporter (VGAT) promoter, where a PET1 promoter (see, e.g., Liu et al. (2010) Nat. Neurosci. 13:1190), a neuropeptide Y (NPY) promoter, a somatostatin (SST) promoter, an arginine vasopressin promoter (see, e.g., Pak et al. (2007) 148:3371), an Ef1a promoter, and a cytomegalovirus early enhancer/chicken β actin (CAG) promoter (see, e.g., Alexopoulou et al. (2008) MBC Cell Biol. 9:2).

Suitable promoters include a myosin light chain-2 (MLC-2) promoter, an α-myosin heavy chain (α-MHC) promoter, a desmin promoter, an AE3 promoter, a cardiac troponin C (cTnC) promoter, and a cardiac acti promoter n. Franz et al. (1997) Cardiovasc. Res. 35:560-566; Robbins et al. (1995) Ann. N.Y. Acad. Sci. 752:492-505; Linn et al. (1995) Circ. Res. 76:584-591; Parmacck et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051. See also, Pacak et al. (2008) Genet Vaccines Ther. 6:13.

### Methods for Producing RAAV Virions

The present disclosure methods for producing a recombinant AAV (rAAV) virion, the method comprising culturing a eukaryotic cell of the present disclosure in a liquid culture medium under conditions such that the rAAV virion is produced by the cell, where the eukaryotic cell comprises: (i) a nucleic acid comprising a nucleotide sequence encoding a variant MAAP of the present disclosure; (ii) nucleic acid(s) comprising nucleotide sequences encoding AAV cap and rep gene products; and (iii) an rAAV.

A method of the present disclosure provides for increased production of rAAV virions from a genetically modified eukaryotic cells of the present disclosure (also referred to herein as a “producer cell”). For example, a method of the present disclosure provides a level of production of rAAV virions that is at least 10%, at least 15%, at least 20%, at least 25%, at least 30%, at least 40%, at least 50%, at least 60%, at least 70%, at least 80%, at least 90%, at least 100% (or two-fold), at least 2.5-fold, at least 5-fold, at least 10-fold, at least 25-fold, at least 50-fold, at least 100-fold, or more than 100-fold, higher than the level of production when the producer cell comprises a nucleotide sequence encoding a wild-type AAV MAAP. In some cases, at least 50%, at least 55%, at least 60%, at least 70%, at least 80%, at least 90%, or more than 90%, of the rAAV virions are secreted into the culture medium in which the producer cell is cultured.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A variant adeno-associated virus (AAV) membrane-associated accessory protein (MAAP) comprising: a) an amino acid sequence having at least 75% amino acid sequence identity to the amino acid sequence of SL01 MAAP or SL35 MAAP;
b) an amino acid sequence having at least 75% amino acid sequence identity to the amino acid sequence of SL30 MAAP or SL31 MAAP; or
c) an amino acid sequence having at least 75% amino acid sequence identity to the amino acid sequence of SL08 MAAP.

Aspect 2. The variant AAV MAAP of aspect 1, wherein the MAAP comprises an amino acid sequence having at least 75% amino acid sequence identity to the amino acid sequence of SL01 MAAP or SL35 MAAP, and wherein amino acid 7 is G or S, amino acid 10 is R or S, amino acid 14 is A or T, amino acid 33 is T or S, amino acid 34 is S or T, amino acid 49 is L or S, amino acid 51 is M or T, amino acid 52 is S or T, amino acid 58 is G or D, amino acid 60 is P or S, amino acid 63 is D or E, amino acid 64 is N or T, amino acid 71 is A or T, and amino acid 73 is S or T.

Aspect 3. The variant AAV MAAP of aspect 1, wherein the MAAP comprises an amino acid sequence having at least 75% amino acid sequence identity to the amino acid sequence of SL30 MAAP or SL31 MAAP, and wherein amino acid 6 is Por Q, amino acid 10 is S or G, amino acid 19 is S or L, amino acid 38 is C or S, amino acid 39 is R or P, and amino acid 51 is T or S.

Aspect 4. The variant AAV MAAP of aspect 1, wherein the MAAP comprises an amino acid sequence having at least 95% amino acid sequence identity to the amino acid sequence of SL01.

Aspect 5. The variant AAV MAAP of aspect 1, wherein the MAAP comprises an amino acid sequence having at least 95% amino acid sequence identity to the amino acid sequence of SL35.

Aspect 6. The variant AAV MAAP of aspect 1, wherein the MAAP comprises an amino acid sequence having at least 95% amino acid sequence identity to the amino acid sequence of SL30.

Aspect 7. The variant AAV MAAP of aspect 1, wherein the MAAP comprises an amino acid sequence having at least 95% amino acid sequence identity to the amino acid sequence of SL31.

Aspect 8. The variant AAV MAAP of aspect 1, wherein the MAAP comprises an amino acid sequence having at least 95% amino acid sequence identity to the amino acid sequence of SL08.

Aspect 9. A nucleic acid comprising a nucleotide sequence encoding a variant AAV MAAP of any one of aspects 1-8.

Aspect 10. The nucleic acid of aspect 9, wherein the nucleotide sequence is operably linked to a transcriptional control element.

Aspect 11. The nucleic acid of aspect 10, wherein the transcriptional control element is a promoter.

Aspect 12. The nucleic acid of aspect 11, wherein the promoter is a cell type-selective promoter.

Aspect 13. The nucleic acid of aspect 11, wherein the promoter is a regulatable promoter.

Aspect 14. An in vitro eukaryotic cell comprising the nucleic acid of any one of aspects 9-13.

Aspect 15. The eukaryotic cell of aspect 14, wherein the nucleic acid is integrated into the genomic DNA of the cell.

Aspect 16. The eukaryotic cell of aspect 14 or aspect 15, further comprising one or more nucleic acids comprising nucleotide sequences encoding AAV cap and rep gene products.

Aspect 17. The eukaryotic cell of aspect 16, wherein the one or more nucleic acids are integrated into the genomic DNA of the cell.

Aspect 18. The eukaryotic cell of any one of aspects 14-17, wherein the eukaryotic is a mammalian cell.

Aspect 19. The eukaryotic cell of any one of aspects 14-17, wherein the eukaryotic is an insect cell.

Aspect 20. The eukaryotic cell of any one of aspects 14-19, wherein the cell further comprises a recombinant adeno-associated virus (rAAV), wherein the rAAV comprises a nucleotide sequence encoding one or more heterologous gene products.

Aspect 21. The eukaryotic cell of aspect 20, wherein at least one of the one or more heterologous gene products is a polypeptide.

Aspect 22. The eukaryotic cell of aspect 20, wherein at least one of the one or more heterologous gene products is a nucleic acid.

Aspect 23. The eukaryotic cell of any one of aspects 20-22, wherein the rAAV comprises a nucleotide sequence encoding a variant capsid polypeptide.

Aspect 24. The eukaryotic cell of aspect 23, wherein the variant capsid polypeptide confers on an rAAV virion increased infectivity of a non-permissive cell, compared to the infectivity of a control rAAV virion that comprises a wild-type capsid of the same serotype.

Aspect 25. A method of producing a recombinant adeno-associated virus (rAAV) virion, the method comprising culturing the cell of any one of aspects 20-24 in a liquid culture medium under conditions such that the rAAV virion is produced.

Aspect 26. A recombinant adeno-associated virus (rAAV) comprising the nucleic acid of any one of aspects 9-13.

Aspect 27. The rAAV of aspect 26, wherein the rAAV comprises a nucleotide sequence encoding one or more heterologous gene products.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like.

### Example 1: Identification and Characterization of MAAP Variants

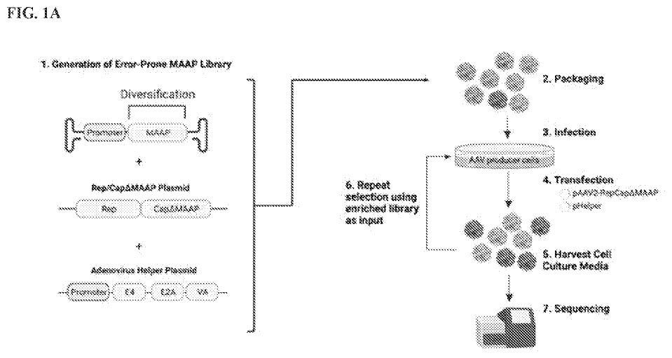

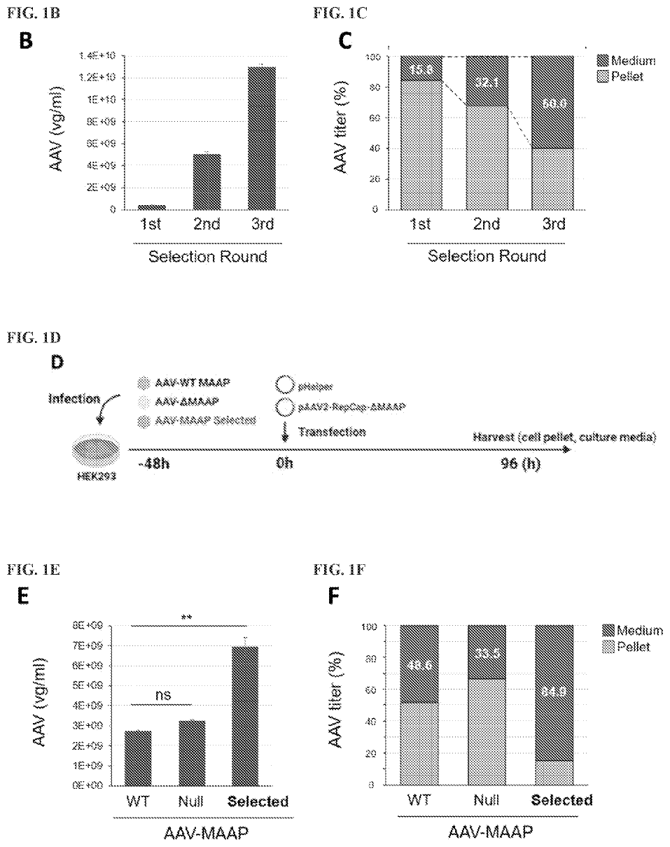

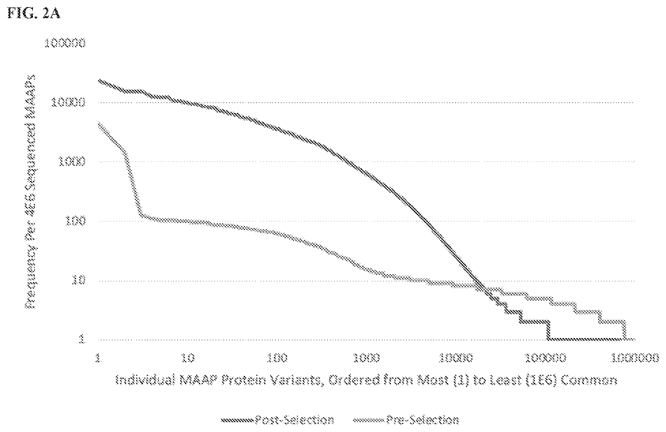

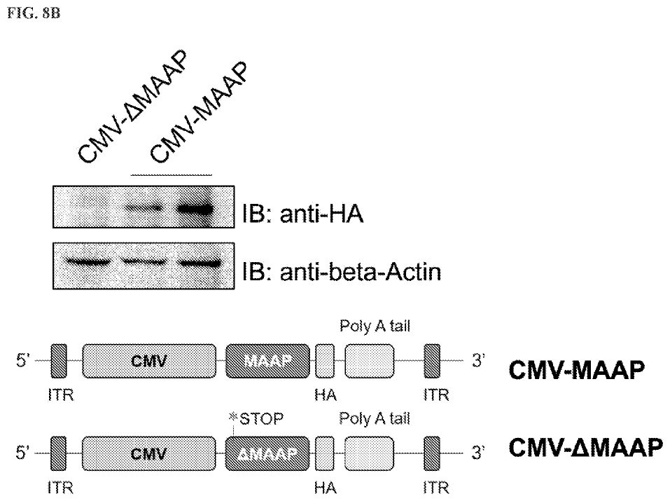

Described herein is an engineering approach based upon directed evolution—the iterative process of sequence diversification and selection of functional gene variants—to identify MAAP sequence variants that confer increased AAV secretion during packaging. First, a library of over 1E6 MAAP variants was generated; the library was then subjected to five rounds of packaging into an AAV2 capsid for which MAAP expression was inactivated without altering the VP1 ORF (AAV2-MAAP-null). Among each iterative packaging round, a progressive increase in both overall titer and ratio of secreted vector genomes conferred by the bulk selected MAAP library population was observed. Next-generation sequencing uncovered common mutational features that were enriched up to over 10,000-fold on the amino acid level. Individual MAAP variants were isolated and systematically tested for effect on recombinant AAV2-MAAP-null packaging in HEK293 cells. This approach is depicted schematically in FIG. 1A-1D and in FIG. 8A-8B.

FIG. 8A-8B: A) Method for inactivation of endogenous MAAP expression from an AAV2 genome, where the method does not affect the VP1 open reading frame. A T-to-A point mutation was introduced into the AAV2 cap region that resulted in the 19^{th} Leucine of MAAP to be mutated to a stop codon but no amino acid change in the overlapping VP1 open reading frame. This recombinant AAV sequence was named “AAV-ΔMAAP.” B) The ΔMAAP sequence containing a C-terminal Human influenza hemagglutinin (HA) tag was transiently transfected into HEK293 cells, and cell lysates were confirmed to lack MAAP protein expression by Western Blot analysis.

FIG. 1A-1F: A) Directed evolution method for identification of MAAP variants that confer increased packaging levels for AAV. B) Head-to-head comparison of total vector genome titer (obtained by qPCR) of the MAAP library bulk population after one, two, or three rounds of AAV2-MAAP-nullpackaging selection relative to the titer after the first round. C) Ratio of secreted vector genomes for samples described in A. D) Schematic for head-to-head comparison of individual MAAP variants. E) Head-to-head comparison of relative total vector genome titer (obtained byqPCR) when packaged into AAV2-MAAP-null containing wild type (WT) MAAP2, no MAAP, or the selected MAAP library bulk population. F) Ratio of secreted AAV for samples in C.

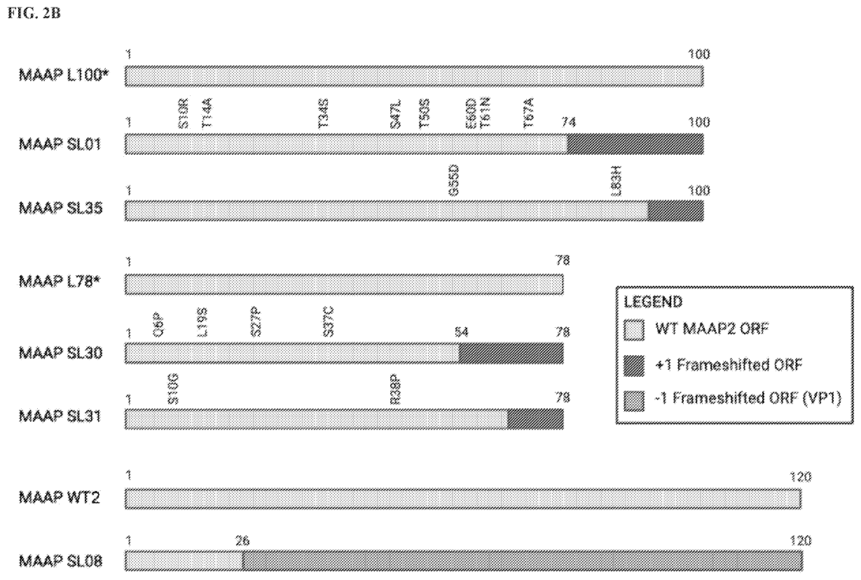

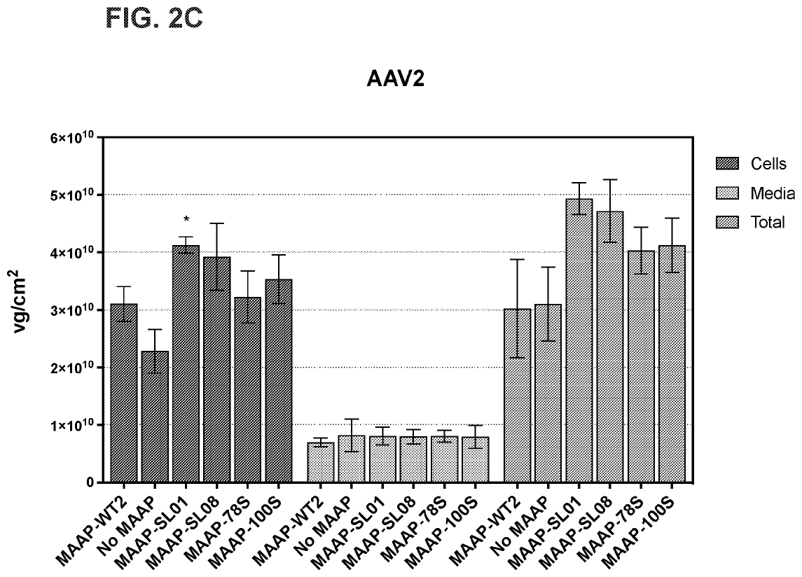

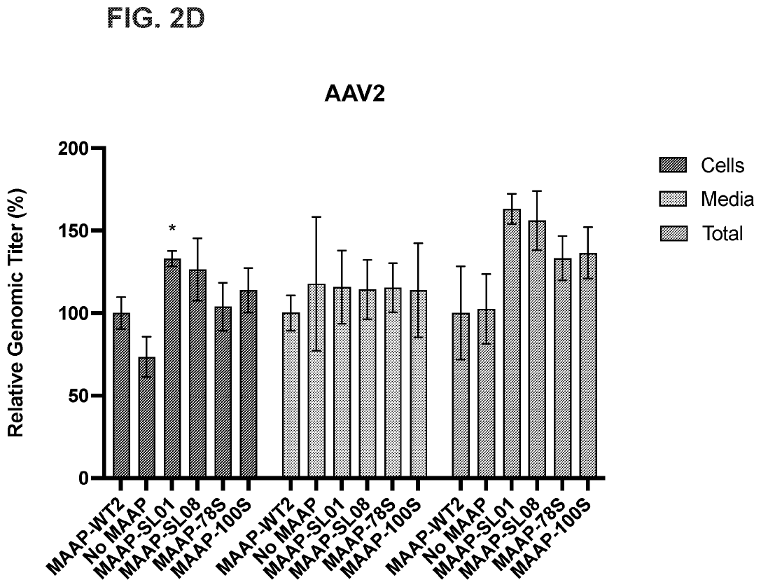

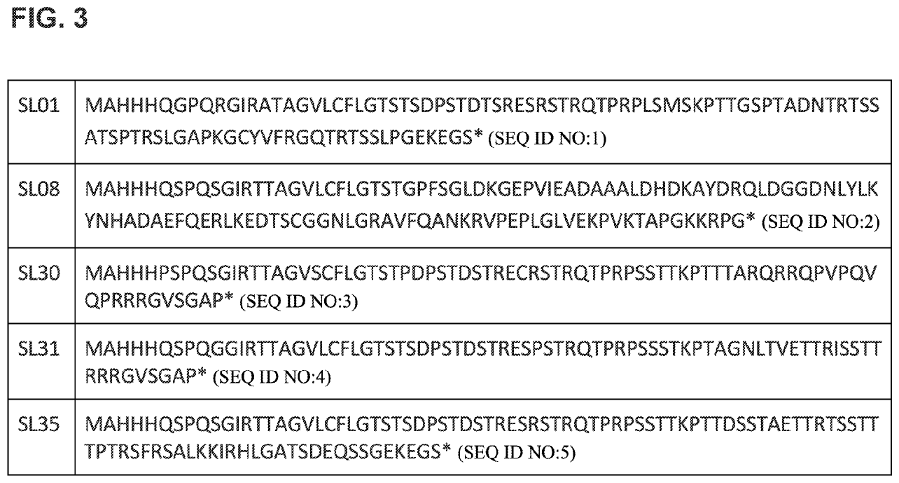

FIG. 2A-2D: Head-to-head packaging comparison of GFP transgene into an AAV2 capsid in HEK293 cells expressing selected leading MAAP variants. A) NGS sequence count of top 1E6 individual MAAP sequences on the amino acid level after diversification and before (orange) or after (blue) four rounds of directed evolution. B) Graphic of five novel MAAP variants chosen from the screen and individually tested for effect on AAV2 packaging. C) AAV2 genomic titer conferred by individual selected MAAP variants when stably expressed from HEK293 cells following lentivirus transduction. Genomic titer was obtained by quantitative PCR (qPCR). Experiment represents six independent biological replicates. D) The data of total vector genome titer in FIG. 2B shown as a percent increase relative to when packaging with AAV2 MAAP (MAAP-WT2).

FIG. 3 provides a table of the amino acid sequences of 5 different variant MAAPs that confer increased AAV secretion during packaging.

### Example 2: Engineered MAAP Variants Confer Increased Vector Genome Production of AAV9 in HEK293 Cells

The cross-serotype activity of MAAP variants were tested in AAV9, another industrially useful AAV serotype. First, the MAAP variants or empty vector (no MAAP control) were stably incorporated into HEK293 cellular genomes using lentivirus transduction and antibiotic selection. Expression of the MAAP variants was driven by a CMV promoter stably integrated in the HEK293 cellular genome. The resulting cell lines were used in a head-to-head packaging comparison of a GFP transgene into an AAV9 capsid encoded by a sequence containing a mutation that ablates the start codon of the endogenous MAAP open reading frame without affecting the overlapping VP1 open reading frame. The engineered SL08 MAAP (MAAP-SL08) variant conferred increased overall recombinant AAV9 production relative to AAV9's endogenous MAAP.

FIG. 4A-4D provide amino acid sequences of four different rationally engineered MAAP derivatives of SL01 and SL08. FIG. 4E-4F provide amino acid sequences of wild-type AAV6 MAAP and wild-type AAV9 MAAP.

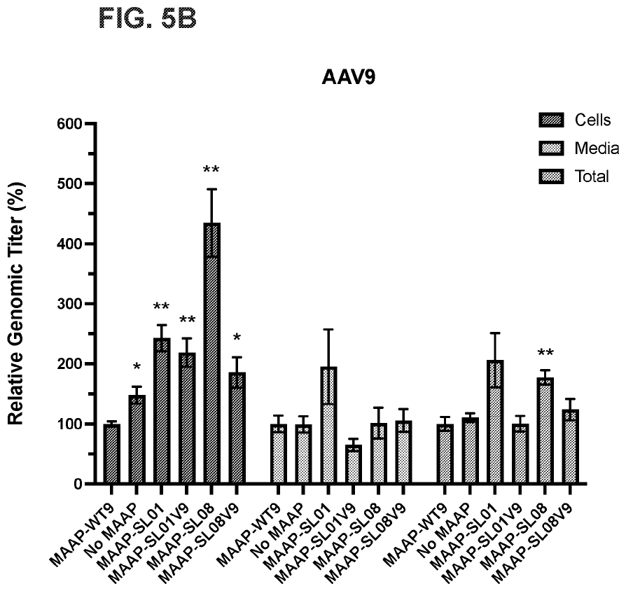

FIG. 5A-B: Head-to-head packaging comparison of a GFP transgene into an AAV9 capsid with selected MAAP variants A) Head-to-head comparison of total vector genome titer (obtained by qPCR) when packaged into AAV9-MAAP-null containing: a wildtype AAV9 MAAP (MAAP-WT9); an empty vector control (No-MAAP); an engineered SL01 MAAP (MAAP-SL01); an engineered SL01 MAAP for which the first 87 amino acids were replaced with the first 87 amino acids of the wildtype AAV9 MAAP (MAAP-SL01V9); an engineered SL08 MAAP (MAAP-SL08); or an engineered SL08 MAAP for which the first 100 C-terminal amino acids of wildtype AAV9 were encoded, followed by a frameshift into 610 N-terminal amino acids of AAV9 VP1 (MAAP-SL08V9). Resulting vector genomes associated with the cell pellet (far left), supernatant (middle), or total (far right) were quantified using qPCR. Each bar represents three independent biological replicates. A student's t test was used to test for statistical significance of each of the other MAAP conditions relative to the wildtype AAV9 MAAP (MAAP-WT9). Statistical significance is indicated with asterisks. B) The data of total vector genome titer in FIG. 5A shown as a percent increase relative to when packaging with AAV9 MAAP (MAAP-WT9).

### Example 3: Engineered MAAP Variants Confer Increased Vector Genome Production and Increased Infectious Titer of AAV6 in HEK293 Cells

The cross-serotype activity of MAAP variants were tested in AAV6, another industrially useful AAV serotype. First, the MAAP variants or empty vector (no MAAP control) were stably incorporated into HEK293 cellular genomes using lentivirus transduction and antibiotic selection. Expression of the MAAP variants was driven by a CMV promoter stably integrated in the HEK293 cellular genome. The resulting cell lines were used in a head-to-head packaging comparison of a GFP transgene into an AAV6 capsid encoded by a sequence containing a mutation that ablates the start codon of the endogenous MAAP open reading frame without affecting the overlapping VP1 open reading frame. The engineered SL08 MAAP (MAAP-SL08) variant conferred increased overall recombinant AAV6 production relative to AAV6's endogenous MAAP.

Next, the post packaging samples were serially diluted onto HEK293 cells in DMEM containing 4% Fetal Bovine Serum (FBS). Infection media was replaced 24 hours post-infection with fresh DMEM containing 10% FBS. Five days post-infection, titers were quantified using flow cytometry analysis of GFP transgene expression. The engineered SL08 MAAP (MAAP-SL08) variant conferred increased overall infectious titer relative to AAV6's endogenous MAAP.

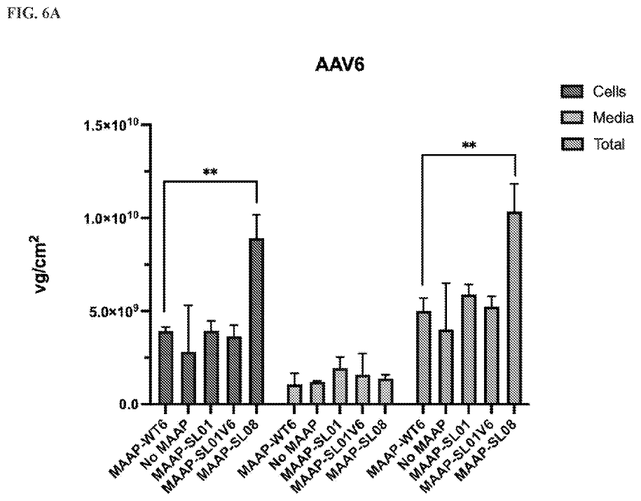

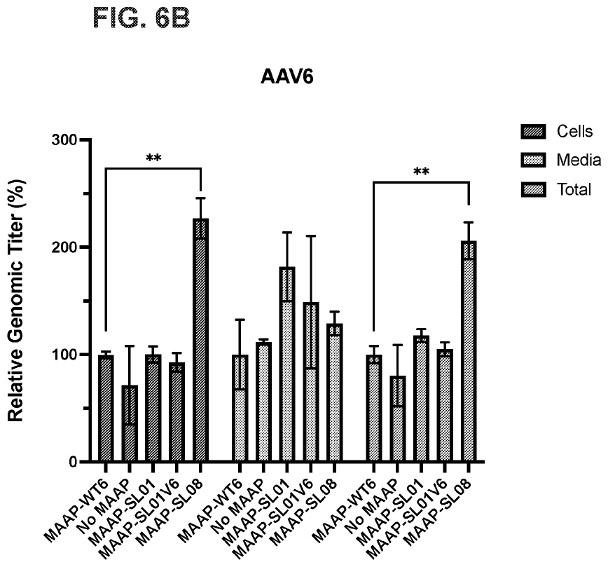

FIG. 6A-6B: Head-to-head packaging comparison of a GFP transgene into an AAV6 capsid with selected MAAP variants A) Head-to-head comparison of total vector genome titer (obtained by qPCR) when packaged into AAV6-MAAP-null containing: a wildtype AAV6 MAAP (MAAP-WT6); an empty vector control (No-MAAP); an engineered SL01 MAAP (MAAP-SL01); an engineered SL01 MAAP for which the first 87 amino acids were replaced with the first 87 amino acids of the wildtype AAV6 MAAP (MAAP-SL01V6); or an engineered SL08 MAAP (MAAP-SL08). Resulting vector genomes associated with the cell pellet (far left), supernatant (middle), or total (far right) were quantified using qPCR. Each bar represents three independent biological replicates. A student's t test was used to test for statistical significance of each of the other MAAP conditions relative to the wildtype AAV6 MAAP (MAAP-WT6). Statistical significance is indicated with asterisks. B) The data of total vector genome titer in FIG. 6A shown as a percent increase relative to when packaging with AAV6 MAAP (MAAP-WT6).

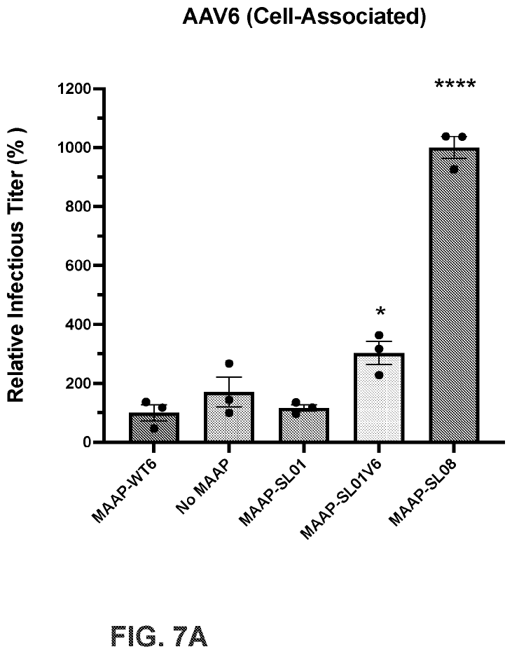

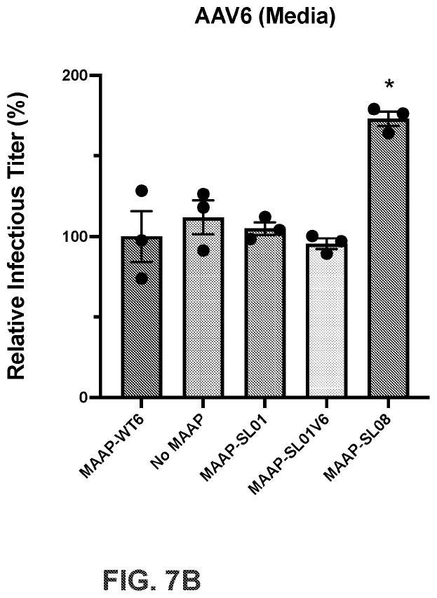

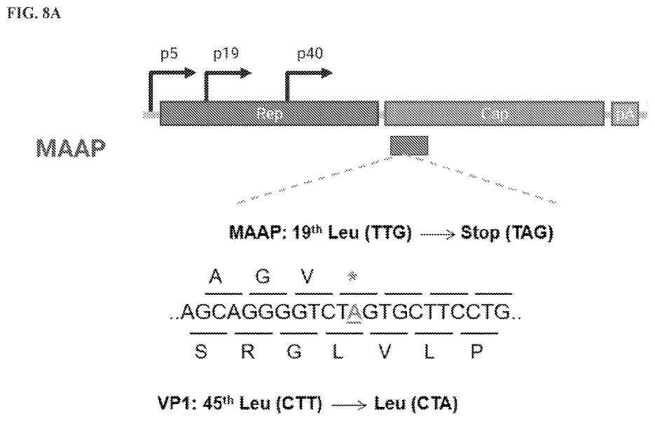

FIG. 7A-7B: Head-to-head infectious titer comparison with selected MAAP variants. A) Head-to-head comparison of cell-associated infectious titer of AAV6-MAAP-null containing: a wildtype AAV6 MAAP (MAAP-WT6); an empty vector control (No-MAAP); an engineered SL01 MAAP (MAAP-SL01); an engineered SL01 MAAP for which the first 87 amino acids were replaced with the first 87 amino acids of the wildtype AAV6 MAAP (MAAP-SL01V6); or an engineered SL08 MAAP (MAAP-SL08). Each bar represents three independent biological replicates. A student's t test was used to test for statistical significance of each of the other MAAP conditions relative to the wildtype AAV6 MAAP (MAAP-WT6). Statistical significance is indicated with asterisks. B) Head-to-head comparison of supernatant-associated infectious titer of AAV6-MAAP-null containing: a wildtype AAV6 MAAP (MAAP-WT6); an empty vector control (No-MAAP); an engineered SL01 MAAP (MAAP-SL01); an engineered SL01 MAAP for which the first 87 amino acids were replaced with the first 87 amino acids of the wildtype AAV6 MAAP (MAAP-SL01V6); or an engineered SL08 MAAP (MAAP-SL08). Each bar represents three independent biological replicates. A student's t test was used to test for statistical significance of each of the other MAAP conditions relative to the wildtype AAV6 MAAP (MAAP-WT6). Statistical significance is indicated with asterisks.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A variant adeno-associated virus (AAV) membrane-associated accessory protein (MAAP) comprising:
a) an amino acid sequence having at least 75% amino acid sequence identity to the amino acid sequence of SL01 or SL35;
b) an amino acid sequence having at least 75% amino acid sequence identity to the amino acid sequence of SL30 or SL31;
c) an amino acid sequence having at least 75% amino acid sequence identity to the amino acid sequence of SL08;
d) an amino acid sequence having at least 75% amino acid sequence identity to the amino acid sequence depicted in FIG. 4A;
e) an amino acid sequence having at least 75% amino acid sequence identity to the amino acid sequence depicted in FIG. 4B;
f) an amino acid sequence having at least 75% amino acid sequence identity to the amino acid sequence depicted in FIG. 4C;
g) an amino acid sequence having at least 75% amino acid sequence identity to the amino acid sequence depicted in FIG. 4D.

2. The variant AAV MAAP of claim 1, wherein the MAAP comprises an amino acid sequence having at least 75% amino acid sequence identity to the amino acid sequence of SL01 or SL35, and wherein amino acid 7 is G or S, amino acid 10 is R or S, amino acid 14 is A or T, amino acid 33 is T or S, amino acid 34 is S or T, amino acid 49 is L or S, amino acid 51 is M or T, amino acid 52 is S or T, amino acid 58 is G or D, amino acid 60 is P or S, amino acid 63 is D or E, amino acid 64 is N or T, amino acid 71 is A or T, and amino acid 73 is S or T.

3. The variant AAV MAAP of claim 1, wherein the MAAP comprises an amino acid sequence having at least 75% amino acid sequence identity to the amino acid sequence of SL30 or SL31, and wherein amino acid 6 is P or Q, amino acid 10 is S or G, amino acid 19 is S or L, amino acid 38 is C or S, amino acid 39 is R or P, and amino acid 51 is T or S.

4. The variant AAV MAAP of claim 1, wherein the MAAP comprises an amino acid sequence having at least 95% amino acid sequence identity to the amino acid sequence of SL01.

5. The variant AAV MAAP of claim 1, wherein the MAAP comprises:
a) an amino acid sequence having at least 95% amino acid sequence identity to the amino acid sequence of SL35;
b) an amino acid sequence having at least 95% amino acid sequence identity to the amino acid sequence of SL30;
c) an amino acid sequence having at least 95% amino acid sequence identity to the amino acid sequence of SL31; or
d) an amino acid sequence having at least 95% amino acid sequence identity to the amino acid sequence of SL08

6. The variant AAV MAAP of claim 1, wherein the MAAP comprises: a) an amino acid sequence having at least 95% amino acid sequence identity to: i) the amino acid sequence MEPRNPKPTSKSRTTAGVWCFLATSTSDPSTDSTRGSPSTRRMQRPSSTTRPTTSSSKRV TIRTCGITTPTPSFRSVCKKIRLLGAT (SEQ ID NO:7); or ii) the amino acid sequence MEPLNPRQINNIKTTLEVLCFRVTNTLDPATDSTRGSRSTQQTRRPSSTTRPTTSSSRPET TRTSSTTTPTPSSRSGSKKIRLLGAT (SEQ ID NO:10); and b) the amino acid sequence RTSSLPGEKEGS (SEQ ID NO:8).

7. The variant AAV MAAP of claim 1, wherein the MAAP comprises: a) an amino acid sequence having at least 95% amino acid sequence identity to the amino acid sequence: MEPRNPKPTSKSRTTAGVWCFLATSTSDPSTDSTRGSPSTRRMQRPSSTTRPTTSSSKRV TIRTCGITTPTPSFRSVCKKIRLLGATSGEQSSRPRRGFS (SEQ ID NO:13); and b) an amino acid sequence having at least 95% amino acid sequence identity to the amino acid sequence:
(SEQ ID NO: 18)
PFGLVEEGAKTAPGKKRPVEQSPQEPDSSSGIGKTGQQPAKKRLNFGQTG
DSESVPDPQPLGEPPATPAAVGPTTMASGGGAPMADNNEGADGVGNASGN
WHCDSTWLGDRVITTSTRTWALPTYNNHLYKQISSASTGASNDNHYFGYS
TPWGYFDFNRFHCHFSPRDWQRLINNNWGFRPKRLNFKLFNIQVKEVTTN
DGVTTIANNLTSTVQVESDSEYQLPYVLGSAHQGCLPPFPADVFMIPQYG
YLTLNNGSQAVGRSSFYCLEYFPSQMLRTGNNFTFSYTFEDVPFHSSYAH
SQSLDRLMNPLIDQYLYYLNRTQNQSGSAQNKDLLFSRGSPAGMSVQPKN
WLPGPCYRQQRVSKTKTDNNNSNFTWTGASKYNLNGRESIINPGTAMASH
KDDKDKFFPMSGVMIFGKESAGASNTALDNVMITDEEEIKATNPVATERF
GTVAVNLQSSSTDPATGDVHVMGALPGMVWQDRDVYLQGPIWAKIPHTDG
HFHPSPLMGGFGLKHPPPQILIKNTPVPANPPAEFSATKFASFITQYSTG
QVSVEIEWELQKENSKRWNPEVQYTSNYAKSANVDFTVDNNGLYTEPRPI
GTRYLTRPL.

8. The variant AAV MAAP of claim 1, wherein the MAAP comprises: a) an amino acid sequence having at least 95% amino acid sequence identity to the amino acid sequence: LEPLNPRQINNIKTTLEVLCFRVTNTLDPATDSTRGSRSTQQTRRPSSTTRPTTSSSRPETT RTSSTTTPTPSSRSGSKKIRLLGATSGEQSSRPKRGFL (SEQ ID NO:11); and b) an amino acid sequence having at least 95% amino acid sequence identity to the amino acid sequence:
(SEQ ID NO: 12)
PLGLVEEAAKTAPGKKRPVEQSPQEPDSSAGIGKSGAQPAKKRLNFGQTG
DTESVPDPQPIGEPPAAPSGVGSLTMASGGGAPVADNNEGADGVGSSSGN
WHCDSQWLGDRVITTSTRTWALPTYNNHLYKQISNSTSGGSSNDNAYFGY
STPWGYFDFNRFHCHFSPRDWQRLINNNWGFRPKRLNFKLFNIQVKEVTD
NNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYA
HSQSLDRLMNPLIDQYLYYLSKTINGSGQNQQTLKFSVAGPSNMAVQGRN
YIPGPSYRQQRVSTTVTQNNNSEFAWPGASSWALNGRNSLMNPGPAMASH
KEGEDRFFPLSGSLIFGKQGTGRDNVDADKVMITNEEEIKTTNPVATESY
GQVATNHQSAQAQAQTGWVQNQGILPGMVWQDRDVYLQGPIWAKIPHTDG
NFHPSPLMGGFGMKHPPPQILIKNTPVPADPPTAFNKDKLNSFITQYSTG
QVSVEIEWELQKENSKRWNPEIQYTSNYYKSNNVEFAVNTEGVYSEPRPI
GTRYLTRNL.

9. A nucleic acid comprising a nucleotide sequence encoding a variant AAV MAAP of any one of claims 1-8.

10. The nucleic acid of claim 9, wherein the nucleotide sequence is operably linked to a transcriptional control element.

11. The nucleic acid of claim 10, wherein the transcriptional control element is a promoter.

12. The nucleic acid of claim 11, wherein the promoter is a cell type-selective promoter.

13. The nucleic acid of claim 11, wherein the promoter is a regulatable promoter.

14. An in vitro eukaryotic cell comprising the nucleic acid of any one of claims 9-13.

15. The eukaryotic cell of claim 14, wherein the nucleic acid is integrated into the genomic DNA of the cell.

16. The eukaryotic cell of claim 14 or claim 15, further comprising one or more nucleic acids comprising nucleotide sequences encoding AAV cap and rep gene products.

17. The eukaryotic cell of claim 16, wherein the one or more nucleic acids are integrated into the genomic DNA of the cell.

18. The eukaryotic cell of any one of claims 14-17, wherein the eukaryotic is a mammalian cell.

19. The eukaryotic cell of any one of claims 14-17, wherein the eukaryotic is an insect cell.

20. The eukaryotic cell of any one of claims 14-19, wherein the cell further comprises a recombinant adeno-associated virus (rAAV), wherein the rAAV comprises a nucleotide sequence encoding one or more heterologous gene products.

21. The eukaryotic cell of claim 20, wherein at least one of the one or more heterologous gene products is a polypeptide.

22. The eukaryotic cell of claim 20, wherein at least one of the one or more heterologous gene products is a nucleic acid.

23. The eukaryotic cell of any one of claims 20-22, wherein the rAAV comprises a nucleotide sequence encoding a variant capsid polypeptide.

24. The eukaryotic cell of claim 23, wherein the variant capsid polypeptide confers on an rAAV virion increased infectivity of a non-permissive cell, compared to the infectivity of a control rAAV virion that comprises a wild-type capsid of the same serotype.

25. A method of producing a recombinant adeno-associated virus (rAAV) virion, the method comprising culturing the cell of any one of claims 20-24 in a liquid culture medium under conditions such that the rAAV virion is produced.

26. A recombinant adeno-associated virus (rAAV) comprising the nucleic acid of any one of claims 9-13.

27. The rAAV of claim 26, wherein the rAAV comprises a nucleotide sequence encoding one or more heterologous gene products.
